(19) United States
(12) Patent Application Publication
(10) Pub. No.: US 2024/0173448 A1
(43) Pub. Date: May 30, 2024

(54) Combination Skin and Medical Connector Disinfecting Device

(71) Applicant: Becton, Dickinson and Company, Franklin Lakes, NJ (US)

(72) Inventors: Manish Kumar, Bengaluru (IN); Shishir Prasad, Ramsey, NJ (US); Anne Shim, West Bloomfield, MI (US)

(21) Appl. No.: 18/071,093

(22) Filed: Nov. 29, 2022

(51) Int. Cl. A61L 2/18; A61B 90/70

(52) U.S. Cl. CPC A61L 2/18; A61B 90/70

(57) ABSTRACT

A device, system, and method for aseptic skin treatment and disinfection of medical connectors comprising a pad soaked with a disinfectant material, the pad having a distal face and a proximal face, a handle associated with the proximal face of the pad, the handle configured for grasping by a user to assist in manipulation of the pad for pressing and scrubbing the distal face of the pad against a surface of the skin. According to one design, an opening extends through a portion of the distal face of the pad and at least partially into a body of the pad. The opening can receive at least an end portion of the medical connector therein to disinfect the end portion of the medical connector. Alternatively the pad can be formed from a soft material capable of deforming about the end portion of the medical connector.

### BACKGROUND OF THE INVENTION

### Field of the Invention

The present disclosure relates generally to a device, system, and method for use in both aseptic skin treatment and disinfection of a medical connector. More particularly, the present disclosure relates to a low-cost, ergonomically designed device, system, and method that is used for cleaning and disinfecting both a skin surface and a medical connector that reduces the occurrence of unwanted touch contamination of the disinfectant during use.

### Description of Related Art

Aseptic skin preparation before performing a medical procedure is often performed using a swab soaked in an alcohol disinfectant material. The alcohol swab can be pre-packaged and removed at the time of treatment or the alcohol can be manually applied onto a dry swab by the practitioner prior to being applied to and/or scrubbed against the patient's skin. This manipulation of the alcohol swab can be difficult due to the small size of the swab. Also, this technique does not support aseptic non-touch technique (ANTT) because the alcohol disinfectant can be in advertently contaminated by the practitioner's hand during removal from the packaging, during the manual application of the alcohol to the dry swab, or even during the scrubbing of the patient's skin with the swab. Further still, when using a pre-packaged pad, the user cannot control the amount of disinfectant applied to the skin surface.

After skin preparation, this same swab or a fresh swab, is used to clean a medical connector, such as a needle-free connector (NFC) or a hub. Again, due to the small size of the swab, manipulation thereof is difficult and can result in unwanted contamination of the disinfectant by the practitioner's hand. Further, due to this small size of the swab, it is difficult to adequately clean the end portion of the medical connector without touching the cleaning surface of the swab and/or without touching the end of the connector itself. Thus, unwanted touch contamination of the disinfectant can occur during this cleaning/disinfecting. Also, a practitioner may use multiple swabs to clean both the patient's skin and the medical connector and/or to ensure adequate disinfection, increasing the cost of the procedure.

There is a need in the art for a low-cost, ergonomically designed cleaning and disinfecting device that can be easily manipulated to support ANTT, wherein the user can control the amount of disinfectant released by the pad, and wherein the same device can be used to effectively disinfect both the skin surface and the medical connector.

### SUMMARY OF THE INVENTION

Accordingly, provided are improved devices, systems, and methods for disinfecting both a skin surface and a medical connector with the same device that supports ANTT.

In accordance with one aspect, the present disclosure is directed to a device for aseptic skin treatment and disinfection of medical connectors comprising a pad soaked with a disinfectant material, the pad having a distal face and a proximal face, a handle associated with the proximal face of the pad, the handle configured for grasping by a user to assist in manipulation of the pad for pressing and scrubbing the distal face of the pad against a surface of the skin, and an opening extending through a portion of the distal face of the pad, and at least partially into a body of the pad, the opening configured for receiving at least an end portion of the medical connector therein such that a portion of the pad comes into contact with the medical connector for disinfecting of the medical connector.

The disinfectant material can be any well-known disinfectant material and can include an antimicrobial or antiseptic material. According to one embodiment, the disinfectant material can comprise a mixture of chlorhexidine gluconate and 70% isopropyl alcohol (CHG-IPA). The pad can be formed from any well-known material, such as a foam or sponge material and the amount of disinfectant material is controlled by the user based on the amount of pressure applied to the pad during skin preparation.

According to one design, the handle can have a color that is different from the pad to distinguish the handle from the pad. This will aid in reducing unintended touch contamination of the pad, especially during the removal of the pad from the packaging. According to one embodiment, the pad and handle can comprise a single piece member. Alternatively, the pad and handle can be separate members.

It can be appreciated that the medical connector can be any well-known medical connector, such as a NFC, a hub, and the like. The opening can be sized to receive the end portion of the medical connector and at least a portion of the surrounding sidewall of the medical connector. The medical connector can be cleaned and disinfected by rotating the connector, such as by a simple to and fro motion, within the opening to ensure disinfection of both the end portion and the surrounding sidewall portion of the medical connector.

In accordance with another aspect, the present disclosure is directed to a method for aseptically cleaning a skin surface and disinfecting a medical connector comprising providing a pad soaked with a disinfectant material, the pad including a distal face and a proximal face, a handle associated with the proximal face, and an opening extending through a portion of the distal face of the pad and at least partially into a body of the pad. The method further includes grasping the handle, scrubbing the distal face of the pad against the skin surface to clean and disinfect the skin surface, removing the pad from the skin, and inserting an end portion of the medical connector into the opening such that at least a portion of the connector sidewall is surrounded by and in contact with a wall portion of the pad.

The method includes rotating the medical connector within the opening to disinfect the medical connector. The method also includes pressing the pad against the skin surface to release the disinfectant located within the pad. The method can include the step of removing the pad from a packaging device by grasping the handle. According to one embodiment, the handle can be formed from a color that is different than the pad to assist in distinguishing the handle from the pad during removal from the packaging device. Providing a different color for the handle can be an easy indication to the user of which portion of the device to grasp, thus reducing unintended touch contamination of the pad.

The pad can be formed from any well-known material, such as foam, sponge material and the like. Also, the disinfectant material can comprise any well-known disinfectant material and can also include any well-known an antiseptic material. It can also be appreciated that the method can be performed on any well-known medical connector, such as NFC, a hub, and the like.

In accordance with yet another aspect, the present disclosure is directed to a skin and connector disinfectant system comprising a sterile packaging having a removable closure and a pad soaked with a disinfectant material, the pad including a distal face and a proximal face, a handle associated with the proximal face, and an opening extending through a portion of the distal face of the pad and at least partially into a body of the pad. The handle can be configured for manipulation of the pad to press the proximal face of the pad onto the skin surface for the scrubbing and disinfecting of the skin while preventing unintended touch contamination. The opening can be configured to receive at least an end portion of the connector for the scrubbing and disinfecting of the end portion of the connector with the pad.

According to one embodiment, the pad and handle can comprise a single piece. Alternatively, the pad and handle can be separate members. The pad can be formed from any well-known material, such as foam, sponge material, and the like. The disinfectant material can be any well-known disinfectant material and can include a well-known antimicrobial or antiseptic material.

During use of the pad, at least a portion of the connector sidewall comes into contact with a wall portion of the pad to disinfect the sidewall of the connector. It can be appreciated that the medical connector can be any well-known medical connector, such as a NFC, a hub, and the like. According to one embodiment, the handle can be formed from a color that is different than the pad to assist in distinguishing the handle from the pad during removal from the packaging. This will aid in a reduction of unintended touch contamination of the pad.

In accordance with still another aspect, the present disclosure is directed to a device for aseptic skin treatment and disinfection of medical connectors comprising a pad soaked with a disinfectant material, the pad having a distal face and a proximal face and a handle associated with the proximal face of the pad, the handle configured for grasping by a user to assist in manipulation of the pad for pressing and scrubbing the distal face of the pad against a surface of the skin, wherein the pad is formed from a material from a very soft material that is configured for deforming around at least an end portion of the connector for disinfecting the connector. The pad can have a hardness of approximately 5-90 Shore A, or 10-50 Shore A, or 15-35 Shore A.

According to one embodiment, the disinfectant material can include an antiseptic material. For example the disinfectant material can comprise a mixture of chlorhexidine gluconate and 70% isopropyl alcohol (CHG-IPA). The pad can comprises one of a foam or sponge material, and the amount of disinfectant material released from the pad is controlled by the amount of pressure applied during use of the pad.

According to one embodiment, the handle can have a color that is different from the pad to assist in distinguishing the handle from the pad to reduce unintended touch contamination of the pad.

It can be appreciated that the medical connector can be a needle-free connector, a hub, and the like. According to one design, the pad and handle can comprise a single piece. According to another aspect, the present disclosure is directed to providing a pad soaked with a disinfectant material, the pad including a distal face and a proximal face and providing a handle associated with the proximal face, wherein the pad is formed from a very soft material that is configured for deforming about an end portion of the connector so as to disinfect the connector. The pad can be formed from a material having a hardness of approximately 5-90 Shore A, approximately 10-50 Shore A, or approximately 15-35 Shore A. The method further includes grasping the handle, scrubbing the distal face of the pad against the skin surface to clean and disinfect the skin surface, removing the pad from the skin, and scrubbing the distal face of the pad against the connector such that the pad deforms about at least the end portion of the connector to scrub and disinfect the end portion of the connector.

The method further includes removing the pad from a package by grasping the handle, and wherein the handle is formed from a color that is different than the pad for assisting in distinguishing the handle from the pad during removal from the package to reduce unintended touch contamination of the pad. After removal from the package, the pad is pressed against the skin surface to release the disinfectant located within the pad.

The pad can be formed from any well-known material such as a foam or sponge material and the disinfectant material can include an antiseptic material.

The medical connector can be a needle-free connector, a hub, and the like.

According to still another aspect, the present disclosure is directed to a skin and connector disinfectant system comprising a sterile packaging having a removable closure, and a pad soaked with a disinfectant material, the pad including a distal face and a proximal face, a handle associated with the proximal face, wherein the pad is formed from a very soft material, such as a material having a hardness of approximately 5-90 Shore A, 10-50 Shore A, or 15-35 Shore A, and is configured for deforming around an end portion of the connector, wherein the handle is configured for manipulation of the pad to press the distal face of the pad onto the skin surface for scrubbing and disinfecting of the skin while preventing unintended touch contamination, and wherein the distal face of the pad is configured to be pressed against the end portion of the connector such that the distal face of the pad deforms about at least the end portion of the connector to scrub and disinfect the end portion of the connector.

According to one embodiment, the pad and handle can be formed from a single piece, the pad can be formed from a foam or sponge material or any other well-known material, and the disinfectant material can include an antiseptic material. The medical connector can be any known type of medical connector, such as a needle-free connector, a hub, and the like. According to one embodiment, the handle can be formed from a color that is different than the pad to assist in distinguishing the handle from the pad during removal of the pad from the packaging to reduce unintended touch contamination of the pad.

Additional embodiments or aspects of the improved devices, systems, and methods for disinfecting both a skin surface and a medical connector with the same device that supports ANTT are detailed in one or more of the following clauses.

In accordance with an additional embodiment of the present invention, a device for aseptic skin treatment and disinfection of medical connectors includes a pad soaked with a disinfectant material, the pad having a distal face and a proximal face; a handle associated with the proximal face of the pad, the handle configured for grasping by a user to assist in manipulation of the pad for pressing and scrubbing the distal face of the pad against a surface of the skin, wherein the pad is formed from a material having a hardness of approximately 5-90 Shore A and is configured for deforming around at least an end portion of the medical connector for disinfecting the medical connector.

The disinfectant material may include an antiseptic material. In certain configurations, the disinfectant material includes a mixture of chlorhexidine gluconate and 70% isopropyl alcohol (CHG-IPA). The pad may include one of a foam or sponge material, and wherein the disinfectant material released from the pad is controlled by the amount of pressure applied during use of the pad. The handle may have a color that is different from the pad to distinguish the handle from the pad to reduce unintended touch contamination of the pad.

In certain configurations, the medical connector includes one of a needle-free connector and a hub. The pad and handle may be formed in a single piece. Optionally, the material may have a hardness of approximately 10-50 Shore A. The material may have a hardness of approximately 15-35 Shore A.

In accordance with another embodiment of the present invention, a method for aseptically cleaning a skin surface and disinfecting a medical connector includes providing a pad soaked with a disinfectant material, the pad including a distal face and a proximal face and providing a handle associated with the proximal face, wherein the pad is formed from a material having a hardness of approximately 5-90 Shore A and is configured for deforming around an end portion of the medical connector for disinfecting of the medical connector, grasping the handle, and scrubbing the distal face of the pad against the skin surface to clean and disinfect the skin surface. The method may also include removing the pad from the skin, and scrubbing the distal face of the pad against the connector such that the pad deforms about at least the end portion of the medical connector to scrub and disinfect the end portion of the connector.

In certain configurations, the method includes pressing of the pad against the skin surface to release the disinfectant located within the pad. The method may also include removing the pad from a package by grasping the handle, and wherein the handle is formed from a color that is different than the pad for assisting in distinguishing the handle from the pad during removal from the package to reduce unintended touch contamination of the pad. The pad may include one of a foam or sponge material and wherein the disinfectant material includes an antiseptic material. The medical connector includes one of a needle-free connector and a hub. The method may also include providing a material for the pad having a hardness of approximately 10-50 Shore A.

In accordance with another embodiment of the present invention, a skin and connector disinfectant system includes a sterile packaging having a removable closure, and a pad soaked with a disinfectant material, the pad including a distal face and a proximal face and a handle associated with the proximal face, wherein the pad is formed from a material having a hardness of approximately 5-90 Shore A and is configured for deforming around an end portion of the medical connector, wherein the handle is configured for manipulation of the pad to press the distal face of the pad onto the skin surface for scrubbing and disinfecting of the skin while preventing unintended touch contamination, and wherein the distal face of the pad is configured to be pressed against the end portion of the medical connector such that the distal face of the pad deforms about at least the end portion of the connector to scrub and disinfect the end portion of the connector.

In certain embodiments, the pad and handle are a single piece. In other configurations, the pad includes one of a foam or sponge material, and wherein the disinfectant material includes an antiseptic material. The medical connector includes one of a needle-free connector and a hub. The handle is formed from a color that is different than the pad to assist in distinguishing the handle from the pad during removal of the pad from the packaging to reduce unintended touch contamination of the pad. The material has a hardness of approximately 10-50 Shore A. Optionally, the material has a hardness of approximately 15-35 Shore A.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a method/device for disinfecting the skin of a patient in accordance with an embodiment of the prior art.

FIG. 2A is a side perspective view of the device for aseptic skin treatment and disinfection of medical connectors in accordance with an embodiment of the present disclosure.

FIG. 2B is a bottom perspective view of the device of FIG. 2A in accordance with an embodiment of the present disclosure.

FIG. 3 is a perspective view of the device of FIG. 2A being used to for aseptic skin treatment and for disinfecting a medical connector in accordance with an embodiment of the present disclosure.

FIGS. 4A-4F show a sequential method for using the device of FIG. 2A in accordance with an embodiment of the present disclosure.

FIG. 5A is a side perspective view of the device for aseptic skin treatment and disinfection of medical connectors in accordance with another embodiment of the present invention.

FIG. 5B is a bottom perspective view of the device of FIG. 5A in accordance with an embodiment of the present disclosure.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

Other than in the operating examples, or where otherwise indicated, all numbers expressing quantities of ingredients, material properties, and so forth used in the specification and claims and Figures are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used.

For purposes of the description hereinafter, the terms “upper”, “lower”, “right”, “left”. “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

In the following discussion, “distal” refers to a direction generally toward an end of a disinfecting device that is adapted for contact with a patient and/or engagement with a separate device such as a medical connector or hub, and “proximal” refers to the opposite direction of distal, i.e., away from the end of a the disinfecting device adapted for contact with the patient or engagement with the separate device. For purposes of this disclosure, the above-mentioned references are used in the description of the components of a syringe assembly in accordance with the present disclosure.

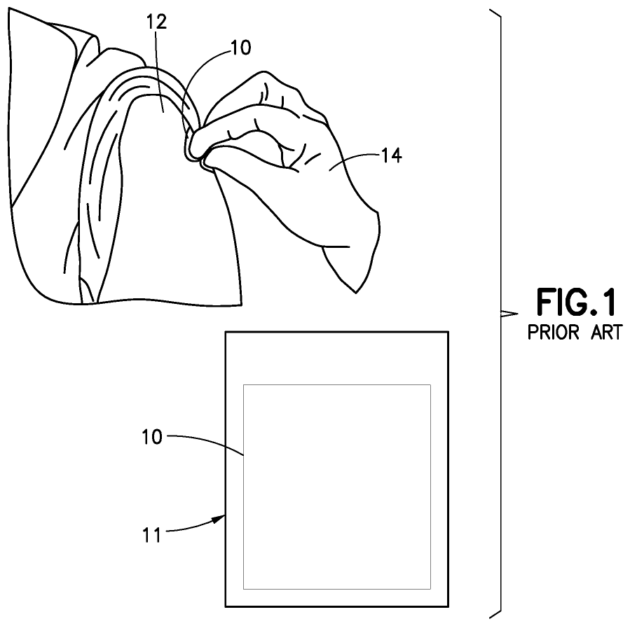

Reference is now made to FIG. 1 which shows the use of an alcohol swab 10 in accordance with the prior art. In order to disinfect the skin of a patient's arm 12, the alcohol swab 10 is removed from a package 11 and applied to the skin of the patient's arm by a medical practitioner 14. The disadvantages of using an alcohol swab, as shown in FIG. 1, is that the swab 10 is small and difficult to manipulate. Thus, it does not support ANTT because the alcohol disinfectant can be in advertently contaminated by the practitioner's hand during removal from the packaging and/or even during the scrubbing of the patient's skin with the swab. Further still, when using a pre-packaged swab 10, the amount of disinfectant applied to the skin surface cannot be controlled. These same risks of contamination are present if the alcohol swab 10 is used to disinfect a medical connector, such as a NFC, a hub, and the like.

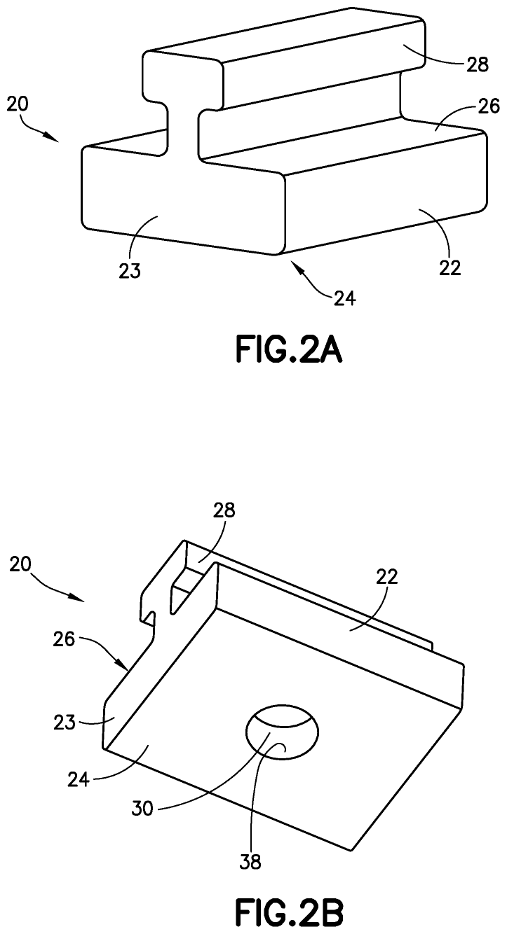

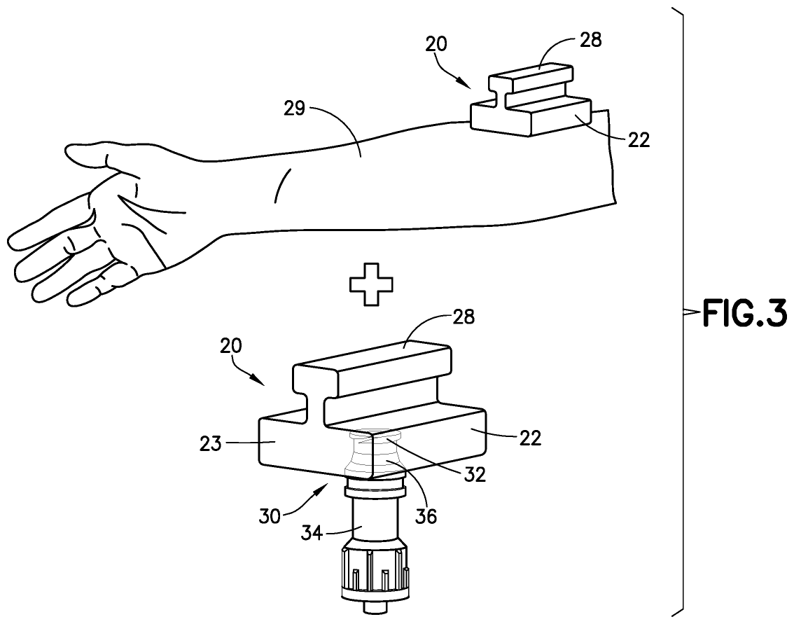

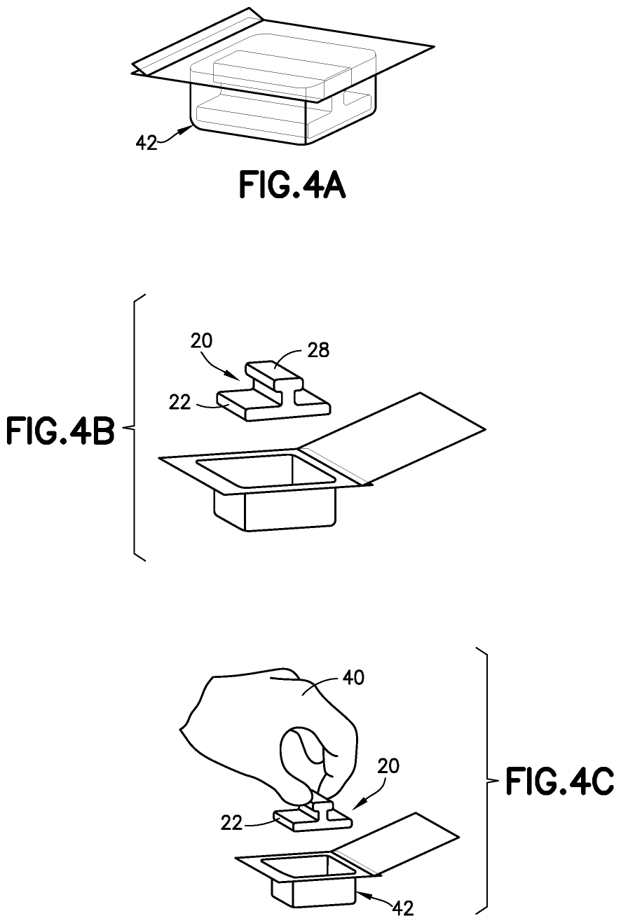

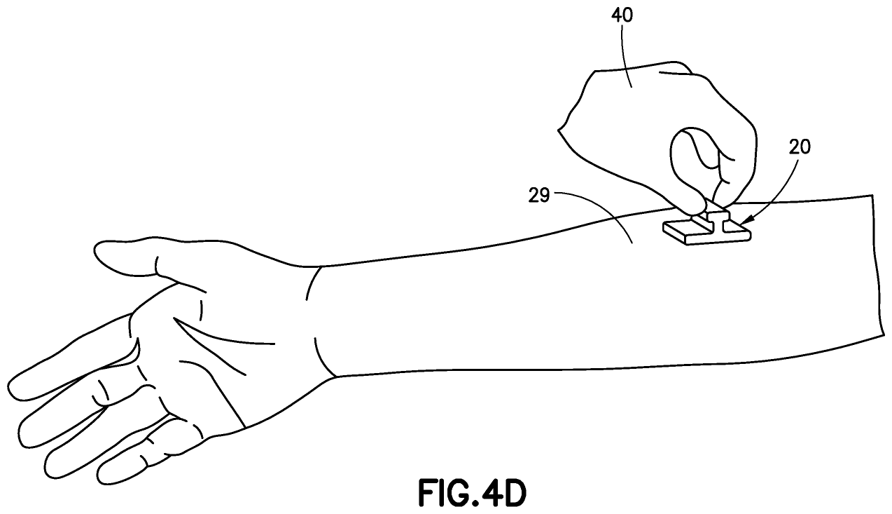

Reference is now made to FIGS. 2A-2B and 3 which show the device 20 for aseptic skin treatment and disinfection of medical connectors in accordance with the present disclosure. The device 20 comprises a pad 22 soaked with a disinfectant material 23. The pad 22 has a distal face 24 and a proximal face 26. A handle 28 is associated with the proximal face 26 of the pad 22. The handle 28 is configured for grasping by a user's hand 40, see FIGS. 4C and 4D, to assist in manipulation of the pad 22 for pressing and scrubbing the distal face 24 of the pad 22 against a surface of a patient's skin 29. An opening 30 is provided that extends at least through the distal face 24 of the pad 22 and partially into a body of the pad 22. The opening 30 is configured for receiving at least an end portion 32 of the medical connector 34 therein such that a portion of the pad 22 comes into contact with the medical connector 34 for disinfecting of the medical connector 34. The disinfectant material 23 can be any well-known disinfectant material and can include an antimicrobial or antiseptic material. According to one embodiment, the disinfectant material 23 can comprise a mixture of CHG-IPA. It can be appreciated that the pad 22 can be formed from any well-known material, such as a foam or sponge material. The amount of disinfectant material 23 that is applied to the patient is controlled by the medical practitioner based on the amount of pressure applied to the pad 22 during skin 29 preparation and/or during disinfection of the medical connector 34.

With continuing reference to FIGS. 2A-2B, and 3 and with further reference to FIGS. 4A-4F, according to one design, the handle 28 can have a color that is different from the pad 22 to distinguish the handle 28 from the pad 22. This will aid in reducing unintended touch contamination of the pad 22, especially during removal of the pad 22 from the sterile packaging 42. According to one embodiment, the pad 22 and handle 28 can comprise a single piece member. Alternatively, the pad 22 and handle 28 can be separate members and the overall device can include additional components (not shown).

It can be appreciated that the medical connector 34 can be any well-known medical connector, such as a NFC, a hub, and the like. The opening 30 can be sized to receive the end portion 32 of the medical connector 34 and at least a portion of the surrounding sidewall 36 of the medical connector 34. The medical connector 32 be cleaned and disinfected by rotating the connector 32, such as by a simple to and fro motion, within the opening 30 so that the end portion 32 and sidewall 36 come into contact with an internal body portion 38 of the pad 22 to ensure disinfection of both the end portion 34 and the surrounding sidewall portion 36 of the medical connector 32.

Reference is now made to FIGS. 4A-4F which show a method and system for aseptically cleaning a skin surface 29 and disinfecting a medical connector 34 comprising providing a pad 22 soaked with a disinfectant material 23. The pad includes a distal face 24 and a proximal face 26. A handle 28 is associated with the proximal face 26, and an opening 30 extends through the distal face 24 of the pad and at least partially into a body portion 38 of the pad 22. The method includes removing the device 20 from the sterile packaging 42 by grasping the handle 28 with a hand 40, scrubbing the distal face 24 of the pad 22 against the skin surface 29 to clean and disinfect the surface of the skin 29, removing the pad 22 from the skin 29, and inserting an end portion 32 of the medical connector 34 into the opening 30 such that at least a portion of the connector sidewall 36 is surrounded by and in contact with an internal wall or body portion 28 of the pad 22.

The method includes rotating the medical connector 34, as shown by arrows 44 in FIG. 4F, within the opening 30 to disinfect the medical connector 34. The method also includes pressing the pad 22 against the surface of the patient's skin 29 to release the disinfectant 23 located within the pad 22. According to one embodiment, the handle 28 can be formed from a color that is different than the pad 22 to assist in distinguishing the handle 28 from the pad 22 during removal from the packaging 42. Providing a different color for the handle 28 can be an easy indication to the medical practitioner/user 40 which portion of the device 20 to grasp, thus reducing unintended touch contamination of the pad 22.

The pad 22 can be formed from any well-known material, such as foam, sponge material and the like. Also, the disinfectant material 23 can comprise any well-known disinfectant material and can also include any well-known an antiseptic material. It can also be appreciated that the method can be performed on any well-known medical connector 34, such as a NFC, a hub, and the like.

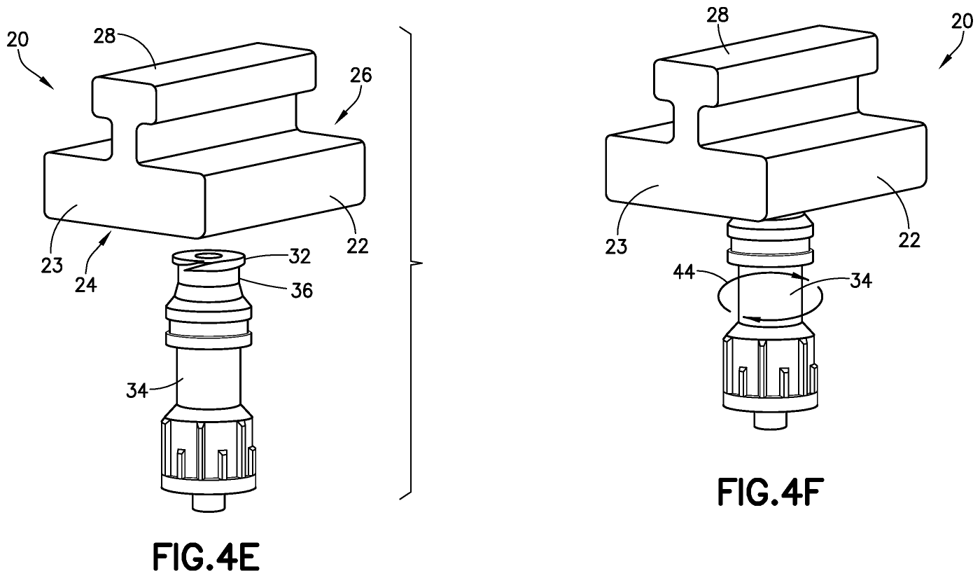

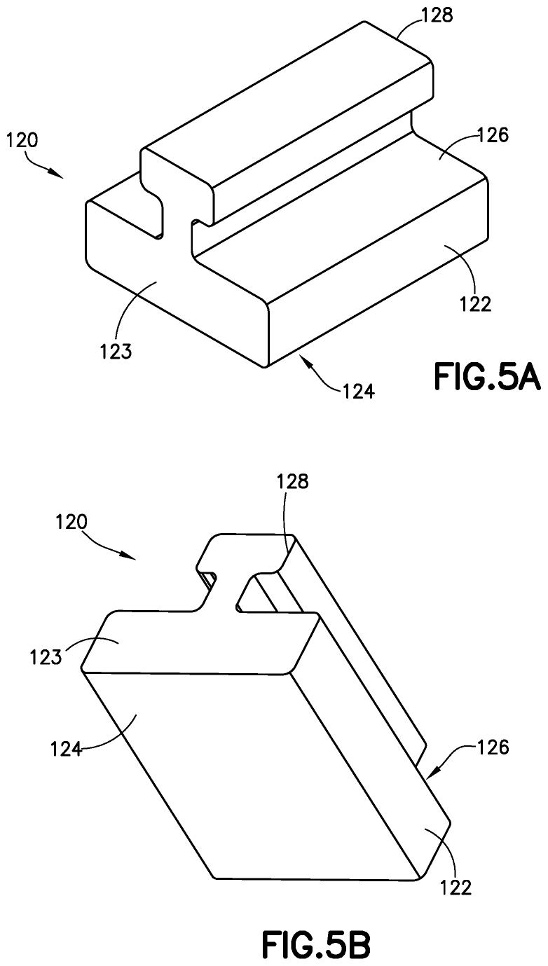

Reference is now made to FIGS. 5A-5B the device 120 for aseptic skin treatment and disinfection of medical connectors in accordance with another embodiment of the present disclosure. It can be appreciated that the device 120 of FIGS. 5A-5B can be used to disinfect both the patient's skin 29 and at least the end portion 32 of the medical connector 34 shown in FIG. 3 and FIG. 4E. The device 120 comprises a pad 122 soaked with a disinfectant material 123. The pad 122 has a distal face 124 and a proximal face 126. A handle 128 is associated with the proximal face 126 of the pad 122. The handle 128 is configured for grasping by a user's hand 40, such as shown in FIGS. 4C and 4D, to assist in manipulation of the pad 122 for pressing and scrubbing the distal face 124 of the pad 122 against a surface of a patient's skin 29, such as shown in FIGS. 3 and 4D. The embodiment shown in FIGS. 5A and 5D differs from the embodiment shown in FIGS. 2A-2B and FIG. 3 in that no opening is provided in the distal face 124 of the pad 122. Instead, the pad 122 is formed from a very soft material that is capable of deforming about at least the end portion 32 of the medical connector 34. The hardness of the material of the pad 122 can be in the range of approximately 5-90 Shore A, approximately 10-50 Shore A, or approximately 15-35 Shore A (ASTM D2240-05).

As discussed above, the disinfectant material 123 can be any well-known disinfectant material and can include an antimicrobial or antiseptic material. According to one embodiment, the disinfectant material 123 can comprise a mixture of CHG-IPA. It can be appreciated that the pad 122 can be formed from any well-known material, such as a foam or sponge material. The amount of disinfectant material 123 that is applied to the patient is controlled by the medical practitioner based on the amount of pressure applied to the pad 122 during skin 29 preparation and/or during disinfection of the medical connector 34.

With continuing reference to FIGS. 5A-5B, according to one design, the handle 128 can have a color that is different from the pad 122 to distinguish the handle 128 from the pad 122. This will aid in reducing unintended touch contamination of the pad 122. It can be appreciated that the method and system illustrated in FIGS. 4A-4E can be followed when using the device 120 shown in FIGS. 5A-5B. It can also be appreciated that forming the handle 128 from a different color than the pad 122 is especially helpful during removal of the pad 122 from the sterile packaging 42, shown in FIGS. 4A-4C. According to one embodiment, the pad 122 and handle 128 can comprise a single piece member. Alternatively, the pad 122 and handle 128 can be separate members and the overall device can include additional components (not shown). The method of using the device 120 would differ from the method of using device 20 in that, instead of inserting the end 32 of the medical connector 34 into a hole in the pad 22 and rotating the device, the soft pad 122 is capable of deforming about the end 32 of the medical connector 34 and the practitioner can rotate the end 32 of the medical connector 34, manipulate the device 120 so that the pad 122 moves about the end 32 of the medical connector 34, or a combination of both so as to disinfect the end 32 of the medical connector 34.

The pad 122 can be formed from any well-known material, such as foam, sponge material and the like. Also, the disinfectant material 123 can comprise any well-known disinfectant material and can also include any well-known an antiseptic material. It can also be appreciated that the method can be performed on any well-known medical connector 134, such as a NFC, a hub, and the like.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

The invention claimed is:

1. A device for aseptic skin treatment and disinfection of medical connectors comprising:
a pad soaked with a disinfectant material, the pad having a distal face and a proximal face;
a handle associated with the proximal face of the pad, the handle configured for grasping by a user to assist in manipulation of the pad for pressing and scrubbing the distal face of the pad against a surface of the skin; and
an opening extending through a portion of the distal face of the pad and at least partially into a body of the pad, the opening configured for receiving at least an end portion of the medical connector therein such that a portion of the pad comes into contact with the medical connector for disinfecting of the medical connector.

2. The device of claim 1, wherein the disinfectant material includes an antiseptic material.

3. The device of claim 2, wherein the disinfectant material comprises a mixture of chlorhexidine gluconate and 70% isopropyl alcohol (CHG-IPA).

4. The device of claim 1, wherein the pad comprises one of a foam or sponge material, and wherein the disinfectant material released from the pad is controlled by the amount of pressure applied during use of the pad.

5. The device of claim 1, wherein the handle has a color that is different from the pad to distinguish the handle from the pad to reduce unintended touch contamination of the pad.

6. The device of claim 1, wherein the medical connector comprises one of a needle-free connector and a hub.

7. The device of claim 1, wherein the opening is sized to receive the end portion and at least a portion of the surrounding sidewall of the medical connector, and wherein the medical connector is configured to be rotated within the opening to ensure disinfection of both the end portion and the surrounding sidewall portion of the medical connector.

8. The device of claim 1, wherein the pad and handle comprises a single piece.

9. A skin and connector disinfectant system comprising:
a sterile packaging having a removable closure; and
a pad soaked with a disinfectant material, the pad including a distal face and a proximal face, a handle associated with the proximal face, and an opening extending through a portion of the distal face of the pad and at least partially into a body of the pad,
wherein the handle is configured for manipulation of the pad to press the distal face of the pad onto the skin surface for scrubbing and disinfecting of the skin while preventing unintended touch contamination, and
wherein the opening is configured to receive at least an end portion of the medical connector for scrubbing and disinfecting of the end portion of the medical connector with the pad.

10. The system of claim 9, wherein the pad and handle comprises a single piece.

11. The system of claim 9, wherein the pad comprises one of a foam or sponge material, and wherein the disinfectant material includes an antiseptic material.

12. The system of claim 9, wherein at least a portion of the connector sidewall comes into contact with a wall portion of the pad to disinfect the sidewall of the connector.

13. The system of claim 9, wherein the medical connector comprises one of a needle-free connector and a hub.

14. The system of claim 9, wherein the handle is formed from a color that is different than the pad to assist in distinguishing the handle from the pad during removal of the pad from the packaging to reduce unintended touch contamination of the pad.

15. A device for aseptic skin treatment and disinfection of medical connectors comprising:
a pad soaked with a disinfectant material, the pad having a distal face and a proximal face;
a handle associated with the proximal face of the pad, the handle configured for grasping by a user to assist in manipulation of the pad for pressing and scrubbing the distal face of the pad against a surface of the skin,
wherein the pad is formed from a material having a hardness of approximately 5-90 Shore A and is configured for deforming around at least an end portion of the medical connector for disinfecting the medical connector.

16. The device of claim 15, wherein the disinfectant material includes an antiseptic material.

17. The device of claim 16, wherein the disinfectant material comprises a mixture of chlorhexidine gluconate and 70% isopropyl alcohol (CHG-IPA).

18. The device of claim 15, wherein the pad comprises one of a foam or sponge material, and wherein the disinfectant material released from the pad is controlled by the amount of pressure applied during use of the pad.

19. The device of claim 15, wherein the handle has a color that is different from the pad to distinguish the handle from the pad to reduce unintended touch contamination of the pad.

20. The device of claim 15, wherein the medical connector comprises one of a needle-free connector and a hub.
